(19) United States
(12) Patent Application Publication
(10) Pub. No.: US 2022/0189074 A1
(43) Pub. Date: Jun. 16, 2022

(54) SYSTEMS, METHODS, COMPUTING PLATFORMS, AND STORAGE MEDIA FOR AUTOMATICALLY DISPLAYING A VISUALIZATION OF A DESIRED VOLUME OF MATERIAL

(71) Applicant: Caleb Hernandez, Denver, CO (US)

(72) Inventor: Caleb Hernandez, Denver, CO (US)

(21) Appl. No.: 17/526,244

(22) Filed: Nov. 15, 2021

Related U.S. Application Data
(60) Provisional application No. 63/113,809, filed on Nov. 13, 2020.
(60) Provisional application No. 63/144,415, filed on Feb. 1, 2021.

(51) Int. Cl. G06T 11/00; A61B 5/00; G16H 10/60

(52) U.S. Cl. CPC G06T 11/00; A61B 5/742; G06T 2207/30004; G06T 2215/16; G16H 10/60

(57) ABSTRACT

Systems, methods, computing platforms, and storage media for automatically displaying a visualization of a desired volume of material are disclosed. Exemplary implementations may: receive a first measurement of a subject for which a desired material is to be applied; receive a name of the desired material receive a desired concentration of the desired material; receive a use case scenario for an application of the desired material to the subject; calculate, based on the first measurement of the subject, the name of the desired material, the desired concentration, and the use case scenario, a correct volume of the desired material for the application; retrieve, from a database, at least one image associated with the correct volume of the desired material for the application; and display, on an interface, the at least one image associated with the correct volume of the desired material.

### CLAIM OF PRIORITY UNDER 35 U.S.C. § 119

The present Applicant for Patent claims priority to U.S. Provisional Application No. 63/113,809 filed Nov. 13, 2020 and U.S. Provisional Application No. 63/144,415, filed Feb. 1, 2021, both of which have the same inventor as the inventor herein, and which are hereby expressly incorporated by reference herein.

### FIELD OF THE DISCLOSURE

The present disclosure relates to systems, methods, computing platforms, and storage media for automatically displaying a visualization of a desired volume of material.

### BACKGROUND

Solutions and suspensions are difficult to comprehend and work with. Anyone who has taken first level chemistry lab has had to convert units to make solutions and suspensions. A first step in creating these solutions and suspensions is often to take a particular mass unit of a powdered chemical that is mixed into a liquid diluent. The amount (i.e., mass) of the active ingredient in the liquid determines the concentration of the solution or suspension. This ratio of mass unit can change in relation to the liquid. For example, 10 mg of calcium can be diluted in 1 mL, 10 mL or 100 mL of sterile water. This would make a 10 mg/mL, 1 mg/mL and 0.1 mg/mL concentration, respectively.

What also complicates things for those making a solution or suspension is that there are different ways to represent the same information. For example the 10 mg/mL solution can be described as a “1% Calcium Solution” or a “1:100 concentration.” There are also issues where different units of measure can be used, which can also create confusion. For example, Moles, Millimoles, mEq (milliequivalents), IU (international units), and grams are all units of measure that can be used interchangeably when mixing or dispensing various solutions/suspensions. This can create confusion, loss of reference, and lead to errors. As long as humans must mix a mass into a volume of liquid, this challenge/problem will be constant.

This task is further complicated by the various measuring containers on the market. For example, in chemistry there are various sized graduated cylinders and flasks, and in medicine, there are various size syringes. An operator could make a mistake by choosing an incorrectly sized measuring device for a task and then set themselves up for a mistake translating the calculated number or volume they want to use or administer on to the syringe. For example, a user can misplace the decimal point on a syringe even if they have calculated the decimal point correctly on paper. Studies have showed with the mean error for such mistakes is substantial—in some studies, as high as 808%.

Those who are working with solutions and suspension of chemicals are typically doing important work in their field, whether it be industry, academia or medical practice. A mistake within calculation could lead to injury, harm, loss or valuable resources or even loss of life. In some fields, such as medicine, there are high error rates for dosing liquid medicines up to 39% of the time (https://pediatrics.aappublications.org/content/pediatrics/141/3/e20174066.full.pdf).

There is a disconnect—a form of functional illiteracy—that makes it difficult to connect the various abstract tasks (e.g., sequence math conversions/measurements) required to convert values for a specific practical task (drawing a syringe of medicine for a specific sized patient, or mixing the appropriate amount of concrete for a specific job). It is nearly impossible to ensure that the people doing the practical jobs have the academic training to understand many of these tasks, and even when there is academic training (medical) the success rate is not 100%. (https://www.ncbi.nlm.nih.gov/pmc/articles/PMC4129781/pdf/WJEM-3-221.pdf). This functional illiteracy results in human error.

There is the need for a reliable way to confirm the correct amount of a solution or suspension to use for particular tasks across a variety of fields and industries.

### SUMMARY

One aspect of the present disclosure relates to a system configured for automatically displaying a visualization of a desired volume of material. The system may include one or more hardware processors configured by machine-readable instructions. The processor(s) may be configured to receive a first measurement of a subject for which a desired material is to be applied. The processor(s) may be configured to receive a name of the desired material receive a desired concentration of the desired material. The processor(s) may be configured to receive a use case scenario for an application of the desired material to the subject. The processor(s) may be configured to calculate, based on the first measurement of the subject, the name of the desired material, the desired concentration, and the use case scenario, a correct volume of the desired material for the application. The processor(s) may be configured to retrieve, from a database, at least one image associated with the correct volume of the desired material for the application. The processor(s) may be configured to display, on an interface, the at least one image associated with the correct volume of the desired material.

Another aspect of the present disclosure relates to a method for automatically displaying a visualization of a desired volume of material. The method may include receiving a first measurement of a subject for which a desired material is to be applied. The method may include receiving a name of the desired material receive a desired concentration of the desired material. The method may include receiving a use case scenario for an application of the desired material to the subject. The method may include calculating, based on the first measurement of the subject, the name of the desired material, the desired concentration, and the use case scenario, a correct volume of the desired material for the application. The method may include retrieving, from a database, at least one image associated with the correct volume of the desired material for the application. The method may include displaying, on an interface, the at least one image associated with the correct volume of the desired material.

Yet another aspect of the present disclosure relates to a computing platform configured for automatically displaying a visualization of a desired volume of material. The computing platform may include a non-transient computer-readable storage medium having executable instructions embodied thereon. The computing platform may include one or more hardware processors configured to execute the instructions. The processor(s) may execute the instructions to receive a first measurement of a subject for which a desired material is to be applied. The processor(s) may execute the instructions to receive a name of the desired material receive a desired concentration of the desired material. The processor(s) may execute the instructions to receive a use case scenario for an application of the desired material to the subject. The processor(s) may execute the instructions to calculate, based on the first measurement of the subject, the name of the desired material, the desired concentration, and the use case scenario, a correct volume of the desired material for the application. The processor(s) may execute the instructions to retrieve, from a database, at least one image associated with the correct volume of the desired material for the application. The processor(s) may execute the instructions to display, on an interface, the at least one image associated with the correct volume of the desired material.

Still another aspect of the present disclosure relates to a system configured for automatically displaying a visualization of a desired volume of material. The system may include means for receiving a first measurement of a subject for which a desired material is to be applied. The system may include means for receiving a name of the desired material receive a desired concentration of the desired material. The system may include means for receiving a use case scenario for an application of the desired material to the subject. The system may include means for calculating, based on the first measurement of the subject, the name of the desired material, the desired concentration, and the use case scenario, a correct volume of the desired material for the application. The system may include means for retrieving, from a database, at least one image associated with the correct volume of the desired material for the application. The system may include means for displaying, on an interface, the at least one image associated with the correct volume of the desired material.

Even another aspect of the present disclosure relates to a non-transient computer-readable storage medium having instructions embodied thereon, the instructions being executable by one or more processors to perform a method for automatically displaying a visualization of a desired volume of material. The method may include receiving a first measurement of a subject for which a desired material is to be applied. The method may include receiving a name of the desired material receive a desired concentration of the desired material. The method may include receiving a use case scenario for an application of the desired material to the subject. The method may include calculating, based on the first measurement of the subject, the name of the desired material, the desired concentration, and the use case scenario, a correct volume of the desired material for the application. The method may include retrieving, from a database, at least one image associated with the correct volume of the desired material for the application. The method may include displaying, on an interface, the at least one image associated with the correct volume of the desired material.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured for automatically displaying a visualization of a desired volume of material, in accordance with one or more implementations.

FIG. 2 illustrates a method for automatically displaying a visualization of a desired volume of material, in accordance with one or more implementations.

FIGS. 3A, 3B, 3C, and 3D show exemplary interfaces of an application that guides a medical provider through a process of inputting variables related to medication dosing and shows a visual display of a correct volume of medication, in accordance with one or more implementations.

FIGS. 4A and 4B show another set of exemplary interfaces of an application that guide a medical provider through a process of inputting variables related to medication dosing and show a visual display of a correct volume of medication, in accordance with one or more implementations.

FIGS. 5A and 5B show exemplary images that may be displayed to indicate a particular volume and type of medication to be given to a patient, in accordance with one or more implementations.

FIGS. 6-11 depict process-flow diagrams associated with aspects of the present disclosure.

FIG. 12 illustrates a block diagram depicting an exemplary machine that includes a computer system within which a set of instructions can execute for causing a device to perform or execute any one or more of the aspects and/or methodologies of the present disclosure, in accordance with one or more implementations.

FIGS. 13A and 13B illustrate examples of a computing device comprising an application for receiving patient measurements, according to various aspects of the disclosure.

FIG. 14 illustrates an example of a computing device displaying a particular volume and type of medication to be taken by a patient, according to various aspects of the disclosure.

### DETAILED DESCRIPTION

Disclosed herein is a system to enable consistent and repeatable interaction with mass and volume units. Once such system provides a confirmation check system, enabling a user to confirm the user is measuring an accurate volume of material and, in fact, doing their job correctly. Since errors in some industries are seen as “never events” (i.e., events that should never happen because such an error would be catastrophic), such a confirmation check system may prevent such errors. It has been found that system-based interventions like simplification and standardizations are more effective than education at reducing human error. (https://www.ismp.org/resources/high-reliability-organizations-hros-what-they-know-we-dont-part-ii).

The present disclosure addresses the need for a high-reliability tool that can be used in industries where measuring volumes of solutions and suspensions performed frequently by many people and where a mistake can have serious consequences. The present disclosure provides a system that is easy to use in association with various volume measuring devices that are commonly available in the market. The system disclosed herein helps users visualize and have a frame of reference for the volume being measured and how to apply the measured volume of material to their specific tasks. The system further provides relevant information a user may need for the to complete an application of the desired material to a subject. It is contemplated that a combination of the first measurement of a subject, a desired material identifier (e.g., a name of the desired material), the concentration of the desired material, and the use case scenario comprises one such application. It is further contemplated that a use case scenario may identify an application (and/or a reason) for associating the desired material with the subject. In one such association, the subject may receive the desired material. In another such association, the subject may ingest the desired material. Providing a visual to users as a frame of reference may enable users to maintain an understanding of the measurement scales involved, potentially reducing mistakes and improving safety. For example, mcg and mg are two very similar-looking units of measure that differ by several orders of magnitude. Many users are not experts in dealing with or understanding such differing scales, allowing many measurement-related mistakes to occur. In a medical setting these measurement-related mistakes can be particularly dangerous and potentially fatal to patients. The present disclosure may comprise one or more key components of a safety system to help users avoid such measurement-related mistakes.

The systems, methods, and apparatuses of the present disclosure have the flexibility and ease of use to take a very technical and complex thought process and simplify the work based on the desired task. They also allow the user to input the specific variables of their current task so that there is no “trial and error,” as elimination of errors is the goal, and “trial and error,” by definition, makes allowance for errors.

In embodiments, a user may use the system of the present disclosure to mix the appropriate amount of cement for a specific job (e.g., paving) associated with a subject (e.g., a driveway). In embodiments, the system may be implemented as a software application on a smartphone. The user could input a first measurement of the subject. Here, a first measurement may comprise more than one value like the length and the width, or the driveway. The user may input the material to be used or mixed into the software application, and input the job type (i.e., “use case,”), which, in this example, may comprise “driveway cement”. The software application may calculate the correct volume of cement that would be needed to pave a driveway of that particular size. It may then guide the user through a process step-by-step how much cement (kg), water (liters), and hardening agent (oz) they should mix for that task, including the size of the container it should be mixed in (25 gallon) and the type of mixing (electric stirring) and length of time mixing should occur. Several of these measurements and variables (in particular, the solution concentrations and resulting volumes) are items that an inexperienced person may not know. For example, a person may not know how much space all the ingredients, when mixed together, would take up. Such a lack of knowledge leads to the possibility of trial and error instead of a perfect result on the first try.

The system of the present disclosure may also automatically account for the fact that both metric and imperial units are used as measures of various components of the solution. Often times, one manufacturer of a particular component will only list instructions in metric units, while another essential component is made by another manufacturer using imperial units. The present system may automatically convert different units and systems of measurement. The ability to convert unlike units, such as, but not limited to, imperial and metric units, into like units may enable the prevention of costly mistakes, save time, and save money by potentially eliminating the need for additional equipment associated with multiple systems of measurement.

In the cement-related example, the system of the present disclosure may display a visualization of the volume of the total amount of cement to be used for the job having a driveway of a particular size. For example, it may show that eight 25-gallon containers should be used to complete the job. The display may be a picture of eight buckets on a screen of a smartphone. In embodiments, text labels, such as the words “25-gallon” may accompany the actual images of the buckets. Such a visualization may reduce waste of materials.

Other embodiments contemplate the administrations of medicine. The use of medication in patients may require the use of several variables related to patient size and drug solution concentration. Variables related to other items may also be included in the administration of medicine. Additionally, the term: variable” may comprise at least one measurement. Patients are various sizes (e.g., small adult (55 kg), child (12 kg), obese adult (140 kg) and certain medications require a specific ratio of milligrams of medicine per each kilogram of body weight, while taking into account the medicant concentrations (10 mg/mL, 50 mg/mL, or 100 mg/mL). When a particular medical condition requires multiple medications or when there are more than one patient who needs to be treated at one time, the amount of variables, medications, patients, and factors can result in a higher likelihood of human error and could cause harm or death.

The embodiments of the present disclosure may aid users in selecting the appropriate tools for a given task. For example, in medical applications of the present invention, a particular syringe size or volume may be suggested to users based on a calculation involving a medication dose, concentration, and volume. Providing aid to users in tool selection may enable a potential reduction in errors and time wasted and may increase success rates, such as by avoiding the use of an improper syringe size that may lead to an error in dosage measurement.

The presently disclosed systems, methods, and apparatuses for displaying appropriate volumes can assist nurses, doctors, pharmacists, and other medical providers in their practice environments, and are highly advantageous, both for the speed of selecting the correct volume, but also to ensure accuracy.

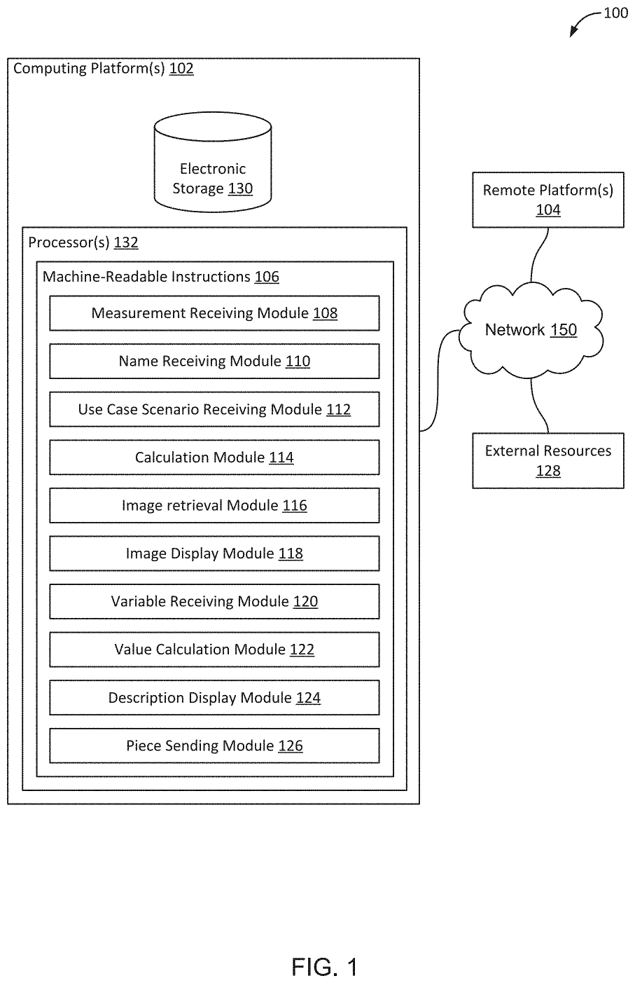

FIG. 1 illustrates a system 100 configured for automatically displaying a visualization of a desired volume of material, in accordance with one or more implementations. In some implementations, system 100 may include one or more servers 102. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Server(s) 102 may be configured by machine-readable instructions 106. Machine-readable instructions 106 may include one or more instruction modules. The instruction modules may include computer program modules. The instruction modules may include one or more of measurement receiving module 108, name receiving module 110, use case scenario receiving module 112, calculation module 114, image retrieval module 116, image display module 118, variable receiving module 120, value calculation module 122, description display module 124, piece sending module 126, and/or other instruction modules.

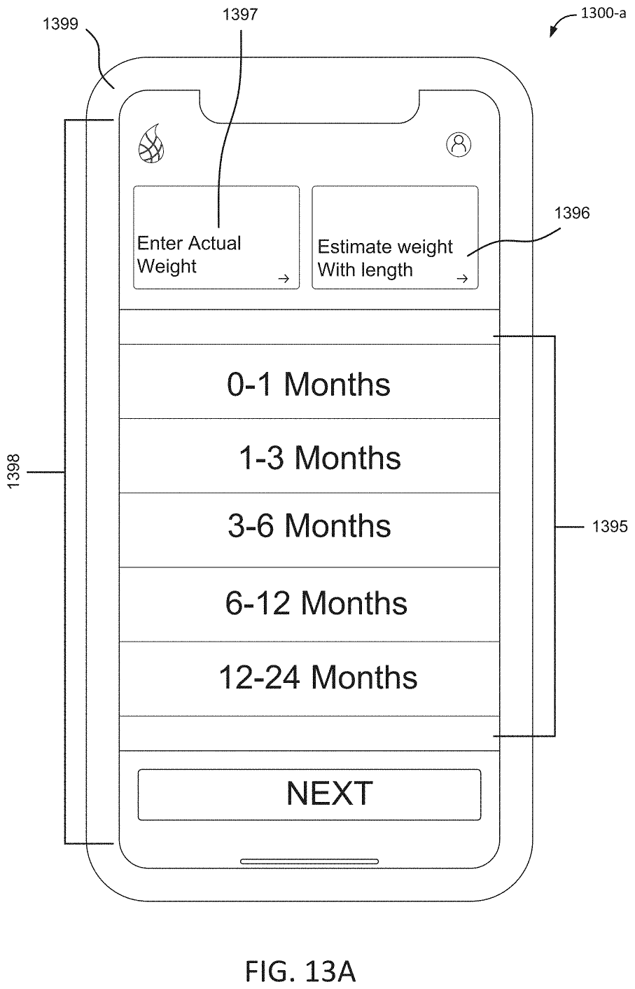

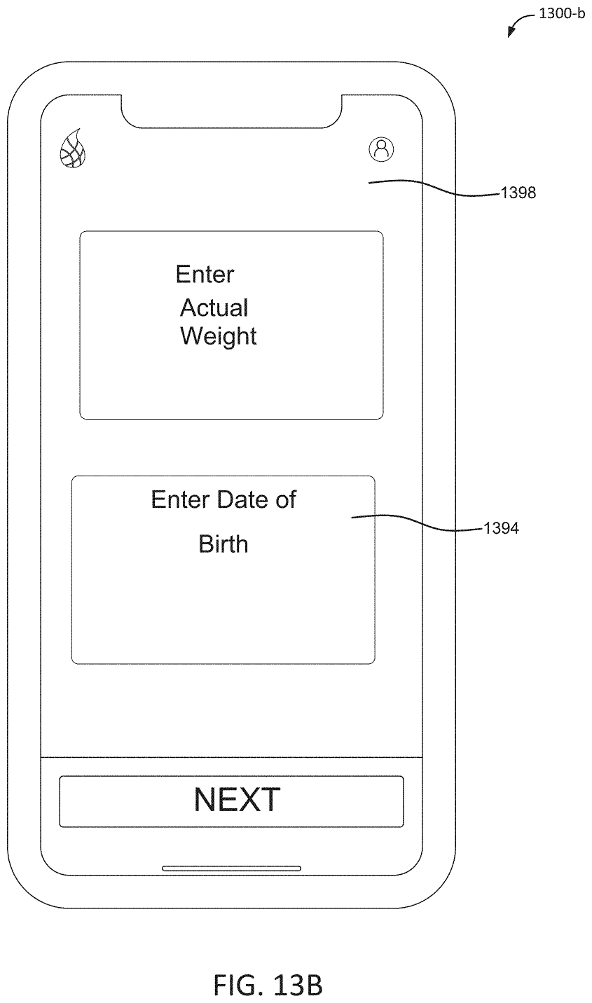

Measurement receiving module 108 may be configured to receive a first measurement of a subject. The subject may comprise any item that can be measured in any way. For example, the driveway in the earlier cement example may comprise a subject. A human medical patient may be another non-limiting example of a subject. Subjects may be associated with a desired material. For example, the driveway subject is associated with the desired material comprising cement in the earlier example and a human patient may be associated with a desired material comprising medicine in a medical example. As seen, the desired material may comprise a desired amount of a material for application to, or association with, the identified subject. Desired materials may comprise a liquid or solid medication, a construction material, a chemical solution, a cooking material, or any other type of material that may be associated with a concentration and a volume. The first measurement of the subject may be a measurement of a patient. However, the term “subject” may refer to any entity (animal, molecular) or item (device, system, area, etc.) associated with the receipt, or application, of the desired material. The measurement of a patient may be a measurement of one of patient length (e.g., inches, centimeters), a patient weight (e.g., pounds, kilograms), and/or patient surface area (e.g., square feet, square meters). Other measurements known in the art are contemplated. The desired material may comprise a medication such as, but not limited to, a liquid oral solution, a liquid injectable solution, a tablet, a pill, or a capsule. Seen in FIG. 13A is one example of a mobile computing device 1399 comprising a software application 1398 which enables a user (e.g. a healthcare provider) to enter a subject (e.g., patient) weight 1397 or a weight estimate 1396. The application 1398 further enables a user to identify the age 1395 of the subject. Alternatively, and as seen in FIG. 13B, the application 1398 may request a date of birth 1394 and the application 1398 may determine the age of the subject.

Measurement receiving module 108 may be configured to receive one or more additional measurements besides the first measurement(s) of a subject. For example, if a first measurement comprise a measurement of a patient's weight, another measurement may comprise the patient's length, height, or surface area. If a first measurement is a length of a physical space, another measurement may be its width, height, or weight.

It is contemplated that the system 100 may comprise an error notification feature. Such a feature may alert a user that a subject (e.g., a patient) should not receive the identified desired material (e.g., a medication). For example, if a child patient is identified as the subject and the desired material is identified as an adult medication, the system may notify the user the medication should not be provided to the patient. In one such embodiment, the information associated with the desired material and subject may be compared to a database to ensure accuracy. For example, a database such as, but not limited to a database associated with eh National Library of Medicine, may be referenced to determine whether an identified medication amount is associated with a patient age and weight. Such a database may comprise a locally-stored database on the device providing the interface or the database may comprise a cloud-based database. Portions of the database may be cloud-based and/or locally-stored. Such alerts and notifications may comprise an audible alarm, a visual notice, and/or may require additional steps to display the correct volume. For example, one such additional step may require an additional device associated with an additional user and/or user account, to approve or otherwise authorize the medication for the identified subject.

Measurement receiving module 108 may receive the first measurement of the subject through a measurement device. Once such measurement device may comprise a hardware measurement device. A hardware measurement device may comprise a camera, laser, scale, accelerometer, or other device, and may be internal or external to the system of the present disclosure. It is contemplated that a hardware measurement device may comprise software and/or firmware. For example, an image of a patient may be taken with a hardware measurement device comprising a camera-enabled mobile computing device (i.e., a smartphone camera), with the image uploaded to the system 100 and the system providing the patient's height upon analyzing the image.

Measurement receiving module 108 may be configured to receive one or more measurements from a device or database that stores subject information, such as subject measurements. For example, an electronic medical record of a patient may be used to provide the measurement receiving module 108 with at least one of a patient length, weight, and surface area. Other modules may also receive information from a device or database that stores subject information. Alternatively, a user may enter the measurement information manually.

Name receiving module 110 may be configured to receive a name of the desired material. It is contemplated that the name of the desired material may comprise an identifier for the material. One such identifier may be an abbreviation associated with the material or may comprise the material name. Such an identifier and/or name may be manually provided. It is further contemplated that the name receiving module 110 may be configured to receive a concentration (e.g., mg/mL) of the desired material. Such a concentration may be referred to herein as a “desired concentration.” In one such example, a user may manually input the desired material name and the concentration of the desired material into the application. It is also contemplated that the computing platform may include optical scanning equipment (e.g., a camera and software) to scan encoded information (e.g., a barcode and/or quick response (QR) code) to obtain the desired information. For example, upon scanning a QR code of a medicine, the application may automatically receive the name of the medicine/desired material and the concentration of the desired material/medicine from a communicatively coupled database.

In another example, machine learning and/or artificial intelligence techniques may be used to interpret an image of a label on a desired material container, and provide information from the label to the name receiving module 110. For example, an imaging device such as, but not limited, to, a camera and software may capture an image and utilize machine learning and/or artificial intelligence techniques to obtain the name, concentration, volume, etc. of the desired material within the container, Such information from the label of the desired material container may also be used in other modules, such as, but not limited to, the calculation module 114. Those of ordinary skill in the art in artificial neural network (ANN) technology will readily appreciate that an ANN model may be trained to interpret the label on a bottle of medication and take information (such as mg/ml, volume, etc.) and incorporate that information into the decision logic described herein.

Use case scenario receiving module 112 may be configured to receive a use case scenario. The use case scenario may be related to the application of the desired material to the subject. For example, the use case scenario may comprise one of a disease state and a treatment protocol. Disease states may also be referred to as “medical conditions” in the present disclosure and disease states and treatment protocols may be referred to together as a “medical use”.

Calculation module 114 (which may be referred to herein as name calculation module) may be configured to calculate, based on (a) the first measurement of the subject, (b) the name of the desired material, (c) the concentration, and (d) the use case scenario, a correct volume (i.e., the proper dose or proper amount of a solution or mixture) of the desired material for the application.

The image retrieval module 116 may be configured to retrieve, from a database, at least one image associated with the correct volume of the desired material for the application. Such an image may comprise a pictogram. The database may be internal or external to the system of the present disclosure. In embodiments, the database may be a library of images that are publicly available, or alternatively, created specifically for the system of the present disclosure. In some implementations, by way of non-limiting example, the at least one image may be of one of a syringe which displays the correct volume of the desired material. Alternatively, the image may display a tablet or a pill having the correct volume of the desired material. The correct volume of tablets, pills, and/or capsules may comprise portions (e.g., half) of such items. In some implementations, the desired material in the at least one image may comprise a color and the color may comprise a first color with the first color comprising a different color as compared to the remainder of the image.

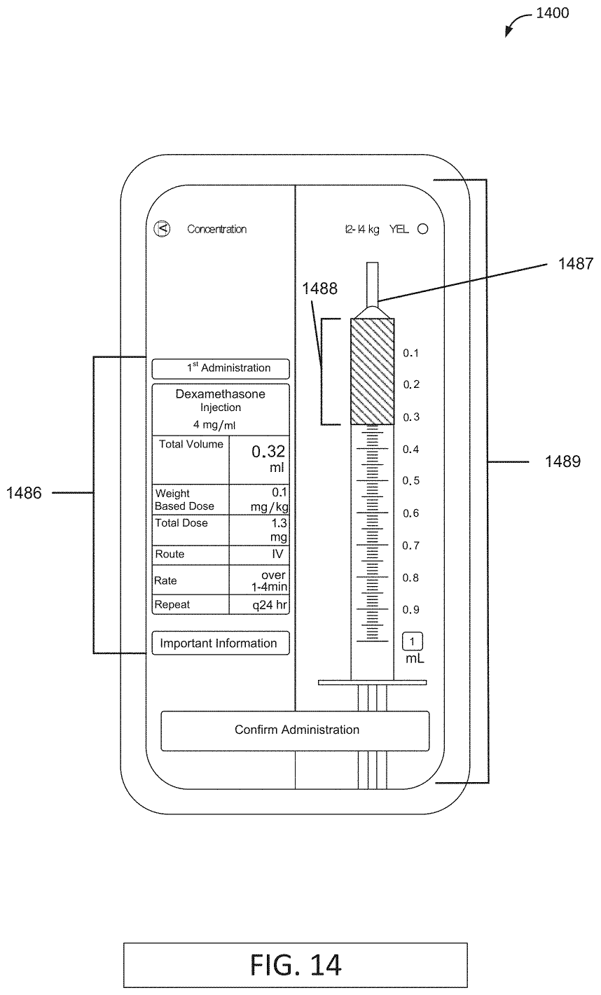

Image display module 118 may be configured to display, on an interface, the at least one image associated with the correct volume of the desired material. A size of the at least one image displayed on the interface may be an actual size of the correct volume. Seen in FIG. 14 is one example of an interface 1400 comprising a mobile computing device/smartphone screen 1489 displaying the at least one image. The at least one image in FIG. 14 comprises the correct volume 1488 of desired material. The desired material in the FIG. 14 example comprises a liquid medicine, or drug, located within a syringe 1487 displayed on the screen. The size of the syringe 1487 and the correct volume 1488 displayed on the screen 1489 may comprise an actual size, enabling a provider to place the actual syringe, used by the provider to give the drug to a patient, next to the syringe 1487 on the screen 1489 to verify the size of the syringe the provider is using is the correct size of syringe and to also verity that the correct volume is the same size as the volume 1488 displayed on the screen 1489. It is contemplated that the correct volume 1488 may comprise a pictogram having feature such as, but not limited to, an identifiable color or pattern (dotted area, slashed/slanted lines, etc.), associated with a subject's age, size, or other subject characteristic. One such pictogram, for example, may comprise a correct volume 1488 having the color pink where pink is associated with a patient having an age less than 3 months or having a weight less than 15 pounds. Characteristics known in the art other than age and size are contemplated. Subject characteristics may comprise any feature associated with a measurement.

The interface 1400 in FIG. 14 further displays a written description 1486 of the correct volume 1488 adjacent to the at least one image of the correct volume 1488 within the syringe 1487. In the FIG. 14 example, the written description 1486 comprises the administration number (i.e., this may comprise the “1st administration” for a particular day or other time period, total administrations of a particular drug, etc.), the drug identifier/name and concentration, current volume, a weight-based dose, a total dose, an administration route for the current volume 1487, a rate of dosage (here, the 3.2 ml will be provided over 1-4 minutes), and a repeat rate, shown as q24 in this example. The above list of items in the written description identify the items shown in FIG. 14 from the top to the bottom of the written description, respectively. In the FIG. 14 display, the correct volume 1488 may comprise an identifiable color. One such identifiable color may comprise a color different from the other colors in the display. Additionally, the color of the correct volume may comprise a color associated with the size of the syringe/pipette and/or a size of the correct volume. For example, a correct volume under 1 ml may comprise a pink color, a correct volume from 1-10 ml may comprise a blue volume, etc.

Variable receiving module 120 may be configured to receive one or more variables related to the subject. Variables may include any number of factors impacting the use case, such as weather, temperature, atmospheric pressure, or physical or chemical requirements associated with a solution or suspension. They may include, in the case of medications, information associated with the administration of the medication and any medical condition associated with the subject. Calculating the correct volume of the desired material may be further based on the one or more variables.

Value calculation module 122 may be configured to calculate one or more estimated values for the subject based on one or more formulas. At least one of the one or more formulas may be used to estimate an ideal body weight of the patient. The calculating of the correct volume may be further based on the one or more estimated values.

Description display module 124 may be configured to display a written description of the correct volume adjacent to the at least one image. The written description of the correct volume may be shown in a plurality of formats.

Piece sending module 126 may be configured to send one or more pieces of information associated with the modules (108, 11, 112, 114, 116, 118, 120, 122, and 124) to an electronic medical records system. For example, the subject measurements from the measurement receiving module and the displayed image from the image display module may be sent to an electronic medical records system.

In some implementations, server(s) 102, client computing platform(s) 104, and/or external resources 128 may be operatively linked via one or more electronic communication links. For example, such electronic communication links may be established, at least in part, via a network such as the Internet and/or other computing networks. It will be appreciated that this is not intended to be limiting, and that the scope of this disclosure includes implementations in which server(s) 102, client computing platform(s) 104, and/or external resources 128 may be operatively linked via some other communication media.

A given client computing platform 104 may include one or more processors configured to execute computer program modules. The computer program modules may be configured to enable a user associated with the given client computing platform 104 to interface with system 100 and/or computing platforms 102 and external resources 128, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 128 may include sources of information outside of system 100, external entities participating with system 100, and/or other resources. In some implementations, some or all of the functionality attributed herein to external resources 128 may be provided by resources included in system 100, and vice versa.

Server(s) 102 may include electronic storage 130, one or more processors 132, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 130 may comprise non-transitory storage media that electronically stores information. The electronic storage media of electronic storage 130 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 may include one or more virtual storage resources (e.g., cloud storage, a virtual private network, and/or other virtual storage resources). Electronic storage 130 may store software algorithms, information determined by processor(s) 132, information received from server(s) 102, information received from client computing platform(s) 104, and/or other information that enables server(s) 102 to function as described herein.

Processor(s) 132 may be configured to provide information processing capabilities in server(s) 102. As such, processor(s) 132 may include one or more of a digital processor, an analog processor, a digital circuit designed to process information, an analog circuit designed to process information, a state machine, and/or other mechanisms for electronically processing information. Although processor(s) 132 is shown in FIG. 1 as a single entity, this is for illustrative purposes only. In some implementations, processor(s) 132 may include a plurality of processing units. These processing units may be physically located within the same device, or processor(s) 132 may represent processing functionality of a plurality of devices operating in coordination. Processor(s) 132 may be configured to execute modules 108, 110, 112, 114, 116, 118, 120, 122, 124, and/or 126, and/or other modules. Processor(s) 132 may be configured to execute modules 108, 110, 112, 114, 116, 118, 120, 122, 124, and/or 126, and/or other modules by software; hardware; firmware; some combination of software, hardware, and/or firmware; and/or other mechanisms for configuring processing capabilities on processor(s) 132. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 108, 110, 112, 114, 116, 118, 120, 122, 124, and/or 126 are illustrated in FIG. 1 as being implemented within a single processing unit, in implementations in which processor(s) 132 includes multiple processing units, one or more of modules 108, 110, 112, 114, 116, 118, 120, 122, 124, and/or 126 may be implemented remotely from the other modules. The description of the functionality provided by the different modules 108, 110, 112, 114, 116, 118, 120, 122, 124, and/or 126 described herein is for illustrative purposes, and is not intended to be limiting, as any of modules 108, 110, 112, 114, 116, 118, 120, 122, 124, and/or 126 may provide more or less functionality than is described. For example, one or more of modules 108, 110, 112, 114, 116, 118, 120, 122, 124, and/or 126 may be eliminated, and some or all of its functionality may be provided by other ones of modules 108, 110, 112, 114, 116, 118, 120, 122, 124, and/or 126. As another example, processor(s) 132 may be configured to execute one or more additional modules that may perform some or all of the functionality attributed below to one of modules 108, 110, 112, 114, 116, 118, 120, 122, 124, and/or 126.

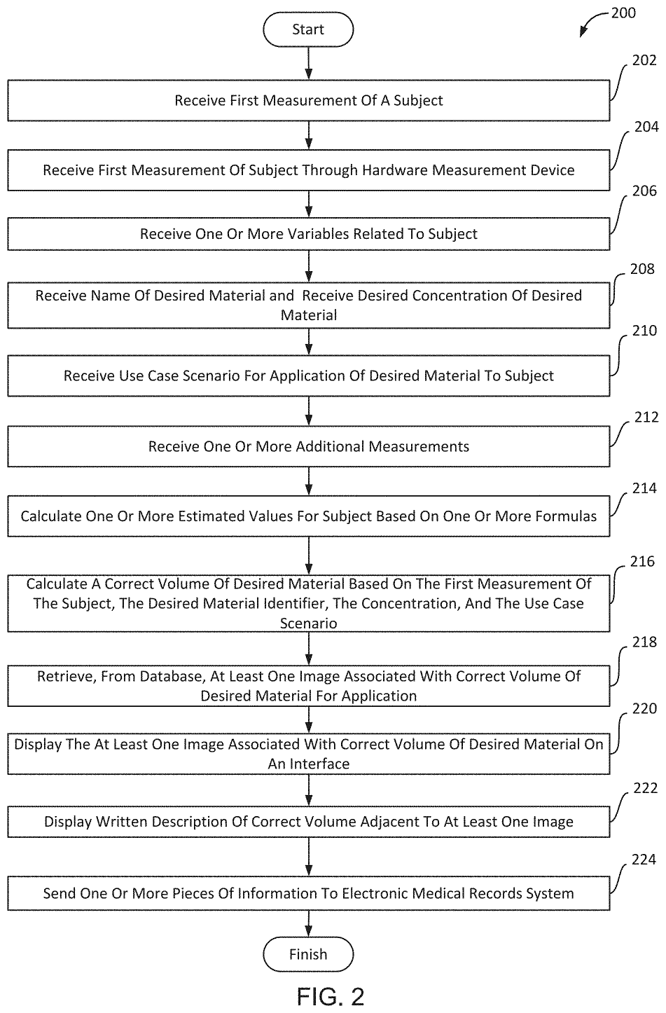

FIG. 2 illustrates a method 200 for automatically displaying a visualization of a desired volume of material, in accordance with one or more implementations. The operations of method 200 presented below are intended to be illustrative. In some implementations, method 200 may be accomplished with one or more additional operations not described, and/or without one or more of the operations discussed. In particular, a subset of the method 200 operations may be executed in some implementations. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

Operation 202 of method 200 may include receiving a first measurement of a subject. As disclosed here, one such subject is associated with an application (i.e. receiving) of a desired material. Operation 202 may be performed by one or more hardware processors configured by machine-readable instructions, including a module that is the same as or similar to measurement receiving module 108, in accordance with one or more implementations.

An operation 204 may comprise receiving the first measurement of the subject through a hardware measurement device. Operation 204 may also be performed by one or more hardware processors configured by machine-readable instructions, including a module that is the same as or similar to measurement receiving module 108, in accordance with one or more implementations.

Operation 206 may comprise receiving one or more variables related to the subject. The calculating of a correct volume of the desired material may be further based on the one or more variables. Operation 206 may be performed by one or more hardware processors configured by machine-readable instructions, including a module that is the same as or similar to variable receiving module 120, in accordance with one or more implementations.

An operation 208 may comprise receiving an identifier associated with the desired material and further receiving a concentration of the desired material. Operation 208 may be performed by one or more hardware processors configured by machine-readable instructions including a module that is the same as, or similar to, name receiving module 110, in accordance with one or more implementations.

An operation 210 may comprise receiving a use case scenario. One such use case scenario comprises a reason the desired material will be provided to the subject. Operation 210 may be performed by one or more hardware processors configured by machine-readable instructions including a module that is the same as or similar to use case scenario receiving module 112, in accordance with one or more implementations.

An operation 212 may include receiving one or more additional measurements. Operation 212 may be performed by one or more hardware processors configured by machine-readable instructions including a module that is the same as or similar to measurement receiving module 108, in accordance with one or more implementations.

An operation 214 may include calculating one or more estimated values for the subject based on one or more formulas. The calculating of the correct volume may be further based on the one or more estimated values. Operation 214 may be performed by one or more hardware processors configured by machine-readable instructions including a module that is the same as or similar to value calculation module 122, in accordance with one or more implementations.

An operation 216 may comprise calculating a correct volume of the desired material, where such calculation is based on (a) the first measurement of the subject, (b) the desired material identifier, (c) the concentration of the desired material, and (d) the use case scenario. Operation 216 may be performed by one or more hardware processors configured by machine-readable instructions including a module that is the same as or similar to Calculation module 114, in accordance with one or more implementations.

An operation 218 may include retrieving, from a database, at least one image associated with the correct volume of the desired material. It is contemplated that the correct volume of the desired material displayed in the image is for the specific application associated with measurements, the subject, use case scenario, and any other variables or information. Operation 218 may be performed by one or more hardware processors configured by machine-readable instructions including a module that is the same as or similar to image retrieval module 116, in accordance with one or more implementations.

An operation 220 may include displaying, on an interface, the at least one image associated with the correct volume of the desired material. Operation 220 may be performed by one or more hardware processors configured by machine-readable instructions including a module that is the same as or similar to image display module 118, in accordance with one or more implementations.

An operation 222 may include displaying a written description of the correct volume adjacent to the at least one image on the interface. One such interface may comprise a smartphone display screen. Other user interfaces known in the art are also contemplated. Operation 222 may be performed by one or more hardware processors configured by machine-readable instructions including a module that is the same as or similar to description display module 124, in accordance with one or more implementations.

An operation 224 may include sending one or more pieces of information to an electronic medical records system. Operation 224 may be performed by one or more hardware processors configured by machine-readable instructions including a module that is the same as or similar to piece sending module 126, in accordance with one or more implementations. In such an embodiment, the mobile computing device screens (e.g., FIGS. 13a-14, etc.) may be integrated in such a medical records system. For example, these screen may be displayed by the system prior to the provider giving the dose to a patient for verification of dosage. Furthermore, although the figures shown herein such, but not limited to FIG. 14, display the correct volume 1487 associated with a syringe, it is contemplated that other dosing mechanisms may be provided. For example, an oral dosing mechanism such a, but not limited, to a cup, may be utilized. Cups in such a system may be color-coded to identify the correct volume. It is further contemplated that the pipette/syringe may be color coded to identify the correct dosing amount. Pipette/syringe sizes may range from 1 ml with a. 25 ml dosing size, 3 ml with a 0.1 ml dosing size, and 5 ml with a 0.2 dosing size. Other sizes known in the art are also contemplated. The timing of such doses may vary as well—e.g., 1×/day, 2×/day, and 4×, and the dose size may vary across one or more doses/day.

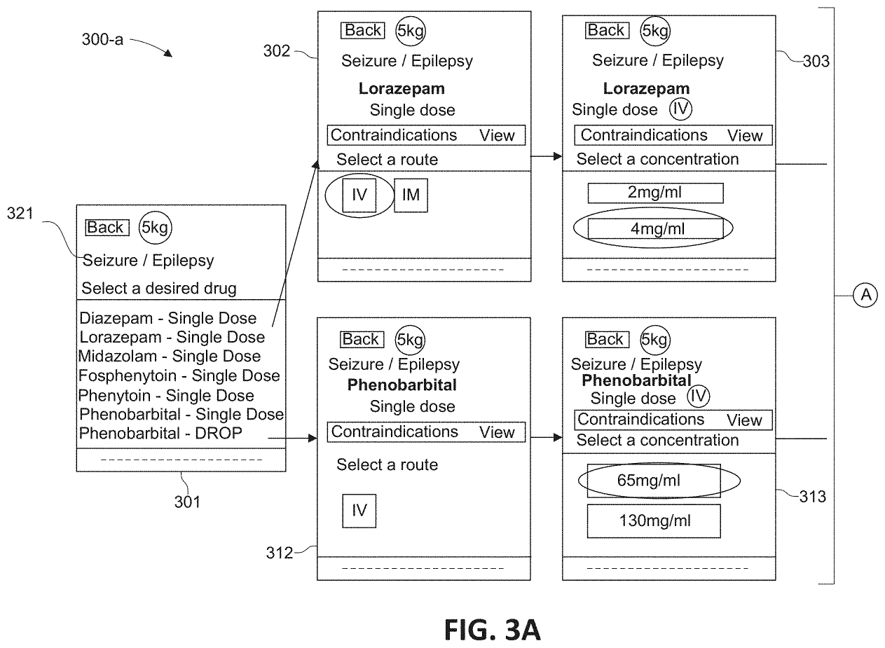

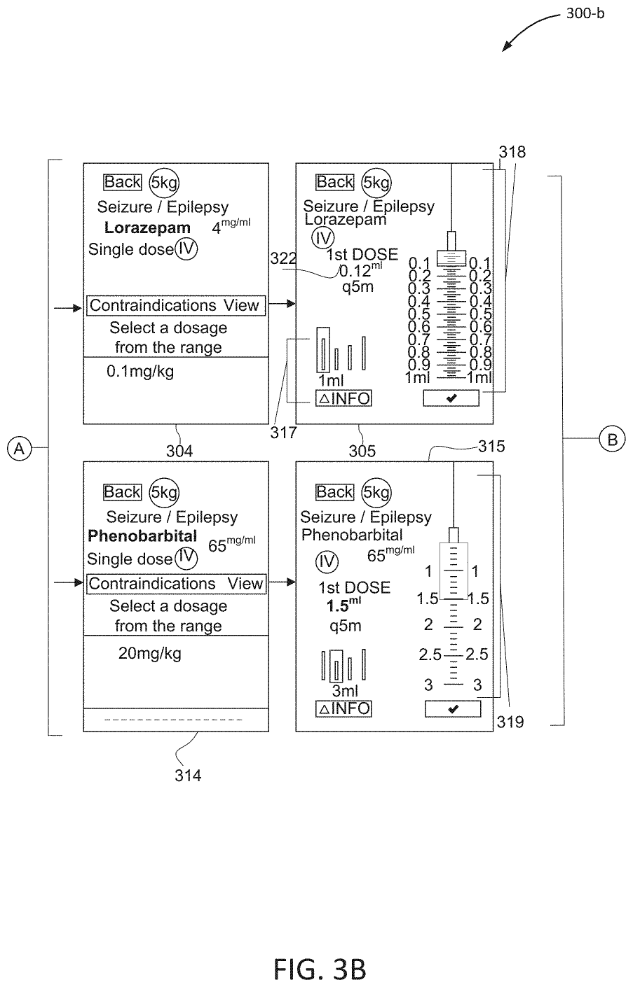

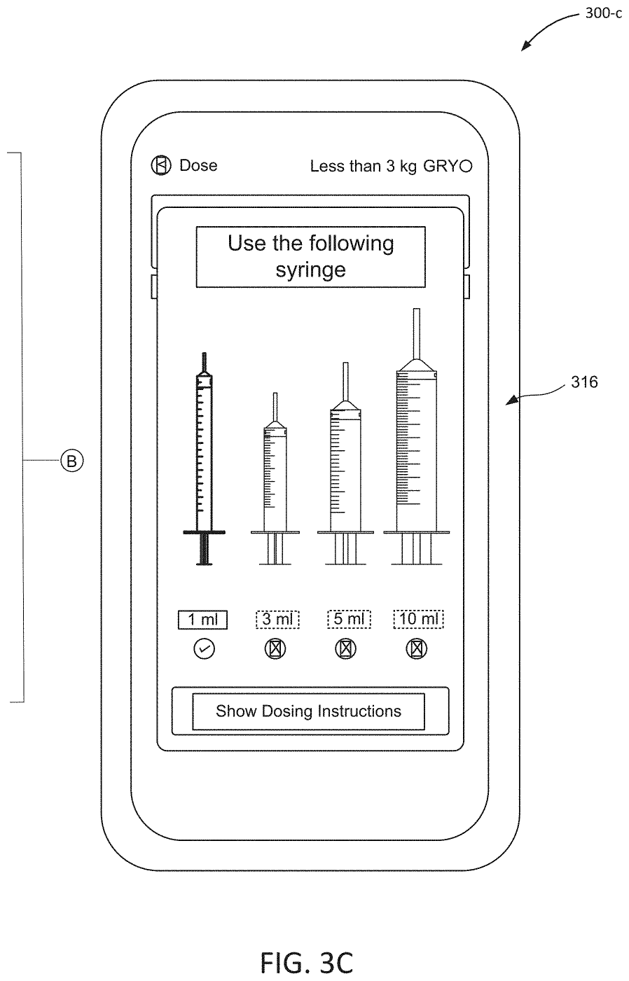

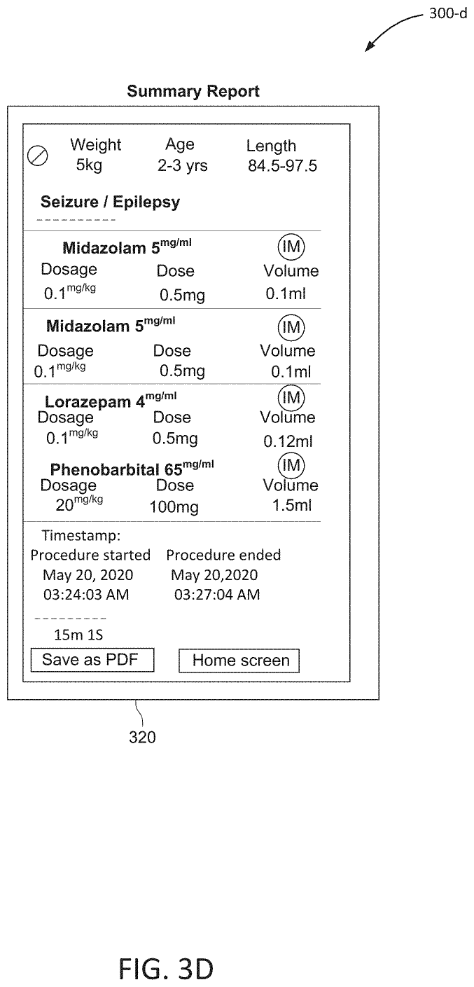

FIGS. 3A-3C illustrate various interface screens 300-a-c of a smartphone application for in accordance with the present disclosure. Process diagram in FIG. 3a begins with a first screen 301. First screen 301 shows a plurality of different types of a desired material comprising a medication (Diazepam, Lorazepam, etc.) for use in association with an already-identified use case 321 (which may have been selected in a prior screen (not shown). Moving now to second screen 302, shown is an example display after a provider selects a particular medication from first screen 301. In second screen 302, Lorazepam was selected from Screen 301. Upon selecting the medication in Screen 301, screen 302 is automatically displayed, which may show options for inputting additional variables. For example, in second screen 302, two routes of administration of the medication are provided: IV (intravenous) and IM (intramuscular). It is contemplated that each of these administration routes or methods have different dosages of the medication. Prior to administering the medication, a medical provider, in one such example, would select the appropriate “route” after third screen 303 is automatically displayed after the administration route variable has been selected (as seen, the IV administration route was selected in this example). In screen 303, the medical provider may be further prompted to select a concentration of the medication, which may also be referred to herein as a drug. As shown in third screen 303, concentrations for the IV route comprise a 2 mg/ml and a 4 mg/ml concentration in this example, which may comprise common manufacturer-provided concentrations. Each of the variables and/or prompts may be stored in a memory or database either internal or external to the system. Upon selecting the concentration in third screen 303, fourth screen 304, as seen in FIG. 3b, may be displayed. In fourth screen 304, one or more dosages of the medicine for the selected concentration may be presented. Here, only one dosage is shown: 0.1 mg/kg. Upon selecting the appropriate dosage, fifth screen 305 may be automatically displayed. Fifth screen 305 may comprise a plurality of different-sized commercially available syringes 317 (with additional information regarding displayed information available in the “INFO” drop-down box). Upon selecting the syringe variable on the screen 305 to align with the syringe size they are using (here, 1 ml), the screen 305 may display a visual indication of the correct volume of the dose, based on each of the received inputs and concentrations. One such visual indication comprises the visual indication 318.

Particular medical conditions or occurrences may require the administration of more than one drug in succession. For example, seizures may require several drugs. FIGS. 3a-3c also display a workflow for a second drug, phenobarbital, which may be required after the implementation of an initial desired material/medicine/drug. Returning now to FIG. 3a, seen is additional second screen 312, which may be automatically displayed after selection of the Phenobarbital menu selection in first screen 301. Additional second screen 312 shows only one option, IV, for the administration route variable. Additionally, third screen 313 shows two possible concentrations: 65 mg/ml and 130 mg/ml of the desired material for the selected IV administration route. Turning now to FIG. 3b, additional fourth screen 314 shows one possible dosage of the identified medicine: 20 mg/kg. Then, on additional fifth screen 315, the provider may select the appropriate size syringe (in this example, 3 ml), and the screen may display a visualization 319 of the correct volume of phenobarbital as it should appear in the syringe.

In some variations, after the fifth screens 305, 315, and as seen in FIG. 3c, a sixth screen 316 may be automatically displayed, where the sixth screen shows photographs of multiple syringe options for the identified desired material, concentration, use case scenario, and variables. A recommended syringe for the dosage displayed in FIG. 3b is with the correct volume is highlighted or otherwise identified as the correct syringe to use in the FIG. 3c display. The system identifies the 1 ml syringe in screen 316 (for a 1 ml dose) as the appropriate syringe to use in for the Lorazepam example above. As seen in screen 305 on FIG. 3b, the 1st dose Lorazepam comprises a 0.12 ml dose. With a 0.12 ml dose, in a 1 ml syringe the dosage will displace the plunger of the syringe along the axial dimension to a greater extent than other syringes. A great axial displacement makes it easier for a user to accurately measure the dose amount and validate the proper dose has been drawn. Screen 316 is exemplary of the types of measurement-tool-size recommendations that may be made according to the embodiments disclosed herein. These calculations and visualizations may be systematized for a specific industry, company, hospital, insurance, etc., and automated, allowing a user of the application to calculate and acquire a proper dosage of drugs quickly and accurately, even in high-pressure situations. Turing now to FIG. 3D, which illustrates an example of an interface screen 300-d, according to an embodiment of the disclosure. As seen, the interface screen 300-d displays an application summary screen 320, where the summary screen 320 may be displayed after the screen 316 shown in FIG. 3C. The summary screen 320 may display all drugs (or other desired material) that may have been dosed (or otherwise provided) for a particular patient/subject during treatment (or for the use case).

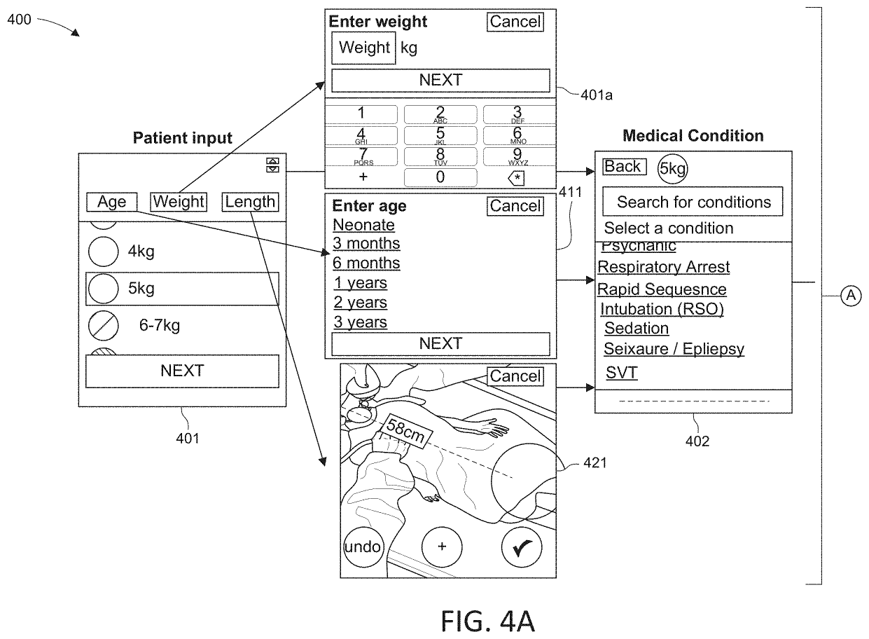

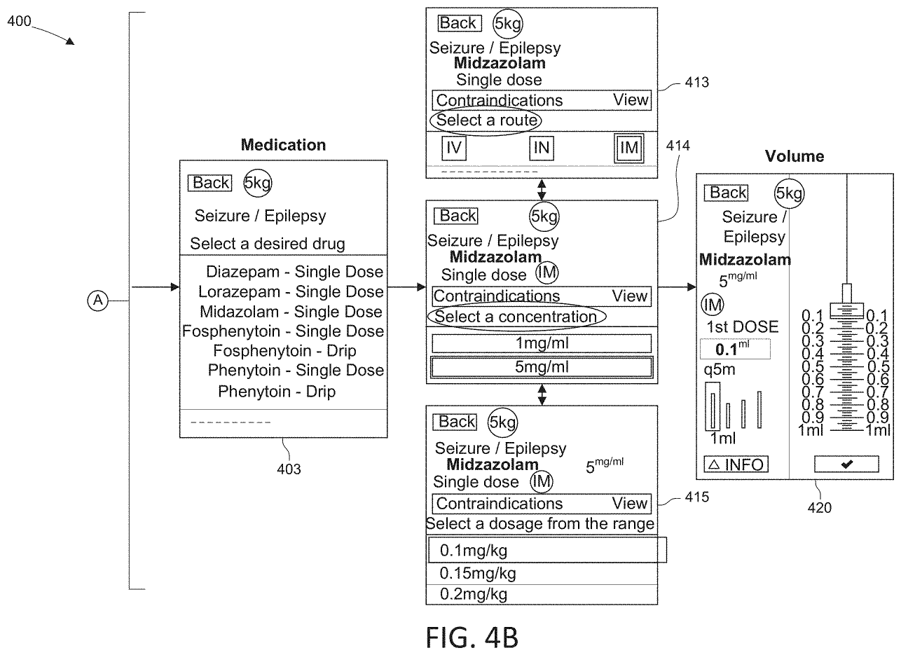

FIGS. 4a and 4b display another workflow 400 of the disclosed system via software application screens. One such workflow starts at screen 401. Screen 401 displays with examples of first measurement(s) for a subject, where the subject in FIG. 4a comprises a patient. There are several measurement examples shown. If the user of the application, which may comprise a medical provider, selects weight as the measurement, the user may enter the weight in screen 401a. If the user selected age as the first measurement, the user may enter or select the age on screen 411. And, if the user selects length as the measurement, they may enter it or use a hardware and/or software measuring device (e.g., a camera configured to measure length) to input length on screen 421. Other ways of entering measurements of a patient and/or any other subject may be used. It is contemplated, and as described herein, the system comprises an age-appropriate weight-based system to help minimize dosing errors.

After entering the proper first measurement, the next screen 402 may be displayed, enabling a user to enter another variable and/or the use case scenario. In the FIG. 4a example, the another variable comprises a medical condition. After selecting the variable, screen 403, as seen in FIG. 4b, may be displayed. Screen 403 may then allow the user (which may comprise a medical provider) to select a medication or other desired material to treat that medical condition/use case scenario. After selecting the medication/desired material in Screen 403, the application may present one or more screens as appropriate for the particular drug (i.e., desired material) and medical condition (i.e., use case). For example, and as shown in screen 414, if Midazolam is selected for the drug associated with a Seizure/Epilepsy use case (or in other cases/drug combinations), the provider may have to identify further variables. The further variables in FIG. 4b comprise: a drug concentration, which, as seen in screen 414, may have the provider select between two concentrations; a drug dose, as seen in screen 415; and a drug administration type, which, as seen in screen 413 may have user select between three different administration routes. Not all drugs may have this exact number of variables, so fewer or more screens may be shown to a user. After the appropriate number of variables screens is displayed, as determined by the user case, desired material, etc., screen 420 may automatically be displayed. The provider may select the correct syringe in screen 420 and the screen may then display the correct volume based on each of the patient measurement, the variables (including medical condition), the drug concentration, and the syringe size.

In embodiments, the system may utilize additional formulas to calculate estimates of other variables. For example, tidal volume (Dose) setting for a ventilator machine is based off length of a patient/subject. A patient length helps to estimate ideal body weight, and known formulas for calculating ideal body weight, and therefore appropriate tidal volume, may be implemented in this disclosure. Thus, knowing ideal body weight can determine tidal volume (how much air goes in or out with each breath) as a ventilator setting, which may also be displayed in a visualization for volume.

In embodiments, the system may alter information or instructions provided to users based on at least one of a use case scenario and a variable associated with a subject, such as a variable received by the variable receiving module 120 of FIG. 1. For example, the suggested medication doses provided to users may differ when treating cardiac bradycardia instead of cardiac arrest, even though the same medication type and administration method is used in treating both conditions.

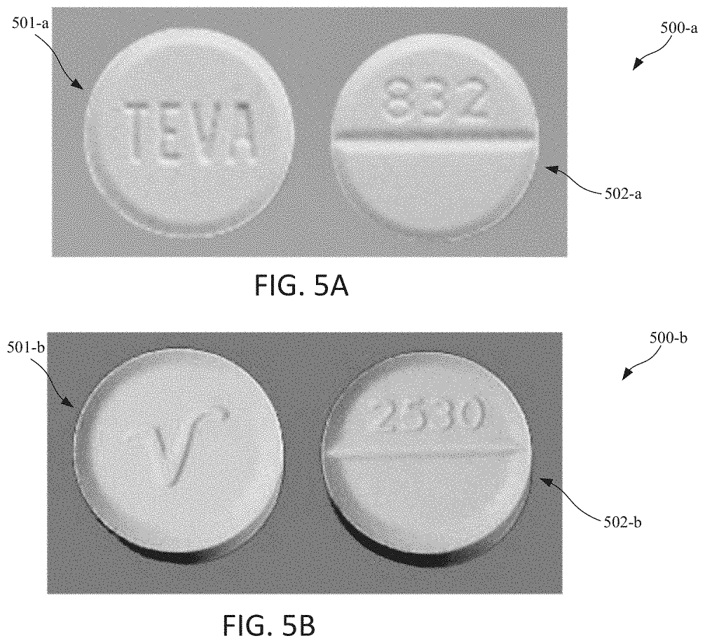

It is contemplated that a correct volume of dosages for medication desired material may be displayed as solid medications such as, but not limited to, tablets and pills. FIGS. 5A and 5B show images of tablets and/or pills 500-a and 500-b, respectively, that may be used as displays of a correct volume of desired material. As see, a front image 501 (e.g., front image 501-a, front image 501-b) and a rear or back image 502 (e.g., rear image 502-a, rear image 502-b) may be displayed for each pill 500. These types of images can be helpful to show not only the number of pills (e.g., 2 pills vs 1 or 1½ pills), but can also show the color and the specific markings (e.g., imprint, such as manufacturer name, a logo, a serial code or another identifier, etc.) the pills have to enable a visual validation by the provider is giving the correct dosage of the correct medication.

In embodiments, the system may further provide timing information to users regarding the application of the desired material to the subject. Timing information may include at least one of drug administration timing, such as IV drip timing, setting or curing timing, such as cement set time, and cooking-related timing, such as proofing time after the application of yeast to a dough in a recipe use case. For example, IV drips may be ordered in mcg/min units, but users may administer IV drips in mL/hour units, which produces a high error rate. The present disclosure may enable a reduction in such error rates by automatically calculating conversions between such units and providing users with a more user-friendly IV drip rate having the appropriate units and a duration of IV drip application. Such timing information may be displayed to users in the application. For example, screens 305 and 315 in FIG. 3b display not only a 0.12 ml and 1.5 ml dosage, abut also display the dosage as “q5m”. Such a term comprises a dosage timing that informs the provider to apply the dosage amount every five minutes.

As discussed herein, part of the system 100 (e.g., a mobile device such as a smart phone or other similar device) may be utilized to interpret an image of a label on a desired material container, with the image obtained using an imaging device (e.g., a camera and software), and the system 100 may obtain information from the label, such as the name, concentration, volume, etc. of the desired material within the container, and provide the information to the name receiving module 110. Such information from the label of the desired material container may also be used in other modules, such as the calculation module 114. And the calculation module 114 may be configured to calculate a correct concentration. The ability to interpret the label to obtain the correct concentration facilitates several functional aspects.

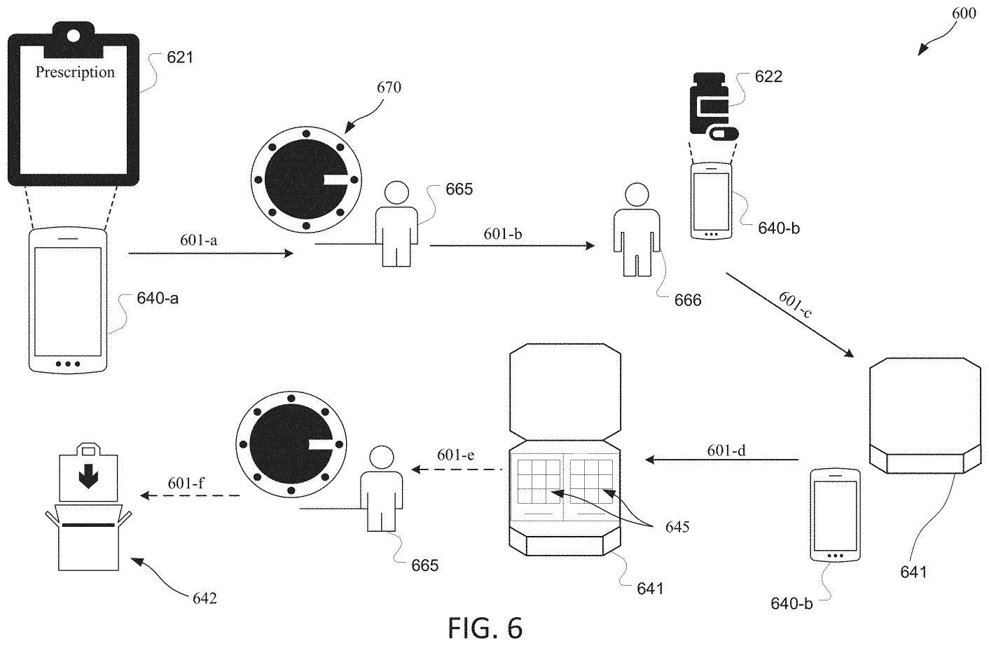

Referring to FIG. 6 for example, shown is a process-flow diagram 600 depicting one or more mobile device(s) 640 (e.g., mobile device 640-a, mobile device 640-b) used in connection with pharmacy ordering; patient pill minding and adherence (e.g., the mobile device 640-b may remind a patient what pills to take); and securing controlled substances in a smart medication security system 641 to prevent the controlled substances from being diverted away from the patient and/or taken by children; thus, preventing misappropriation and accidents.

As seen, a prescription 621 written for a patient (e.g., patient 666) may be scanned using a first mobile device 640-a. In some cases, the prescription 621 may be scanned by the doctor or another medical professional, such as a nurse practitioner. Alternatively, the patient may scan the prescription 621. In either case, at step 601-a, information pertaining to the prescription 621, such as a list of medications, recommended dosage, etc., may be transmitted to a pharmacist 655. Pharmacies typically store medicines in a secure location 670, such as a vault or safe, or an area requiring keycard access. After the pharmacist 655 has filled the prescription, at step 601-b, the patient 666 may collect medicines 622 and scan the container (e.g., bottle, or another storage container) containing the medicines using the mobile device 640-b. In some cases, the mobile device 640-b may be similar or substantially similar to the mobile device 640-a. In other cases, the mobile device 640-b and the mobile device 640-a may be the same mobile device. One or more of the mobile device 640-a and 640-b may comprise a software application, described herein and elsewhere throughout the disclosure. The software application may interface with one or more hardware components (e.g., barcode scanner, camera or imaging device) of the mobile device 640 to scan and/or image a label on the container containing the medicines 622. In some examples, the application may obtain information from the label, such as, but not limited to, name, concentration, volume, etc., of the medicine 622.

At step 601-c, the user or patient 622 may store the container containing the medicine 622 in the smart medication security system 641. The smart medication security system 641 may enable the patient 622 to store the medicines 622 safely and securely. For instance, the smart medication security system 641 may comprising locking features, and may be unlocked using a security PIN or passcode, biometrics (e.g., fingerprint scan, voice recognition, facial ID), a one-time password (OTP) transmitted to the mobile device 640-b, or through any other applicable means.

Although not necessary, in some examples, the smart medication security system 641 may be unlocked (e.g., shown as step 601-d) using the software application stored on the mobile device 640-b. As seen, the smart medication security system 641 comprises a plurality of compartments 645 for storing the user or patient's medication. In some examples, the compartments 645 may be labeled (e.g., with letters or numbers), one for each type of medicine. Further, the application on the mobile device 640-b may designate different compartments for different medicines (e.g., compartment 1 for medicine A, compartment 2 for medicine B, and so on) and instruct the patient 666 to place the container(s) containing the medicines into the assigned compartments.

In some cases, the smart medication security system 641 may transmit a notification to the pharmacist 665, for instance, for an automatic refill, when it determines the patient's medication is running low, to name two non-limiting examples. In such cases, the pharmacist 665 may fill the prescription (e.g., based on the information in prescription 621, previously scanned by mobile device 640-a) and mail it to the patient, at step 601-f. It should be noted that, steps 601-e and/or 601-f may be optional (shown as optional by the dashed lines).

In some other cases, steps 601-b through 601-f may be performed by the pharmacist 665. In this case, the user 666 may be the pharmacist, not the patient. For instance, after the pharmacist fills the prescription 621 at step 601-b, the pharmacist may scan the container containing the medicines 622 using the mobile device 640-b. Information pertaining to the medicine, including the name, dosage, color and/or shape of pills, etc., may be retrieved by the application stored on the mobile device 640-b and linked to the patient and prescription 621. At steps 601-c and 601-d, the pharmacist (i.e., user 666 in this case) may store the medicines 622 in the smart medication security system 641, for instance, in the compartments 645 assigned by the application on the mobile device 640-Sb. In this way, the smart medication security system 641 may be used to store a plurality of medicines, each in a different compartment 645. At step 601-e, the pharmacist 665 may lock the smart medication security system 641 (e.g., via the application on the mobile device 640-b, a physical keycard or fob, a PIN or passcode, etc.) so that it can be delivered to the patient. As seen, at step 601-f, a package 642 containing the smart medication security system 641 may be delivered to the patient's home address. In some cases, the patient or another user (e.g., caregiver) may receive the passcode for unlocking the smart medication security system 641 via an application stored on their mobile device, as a text message, an email, etc. In some cases, this passcode may be a temporary passcode (e.g., only valid for 24 hours after package delivery) and a user/patient may need to update the passcode, set up biometrics authentication (e.g., on the smart medication security system 641 or their user device), and/or set up two-factor authentication to unlock the smart medication security system 641 in the future.

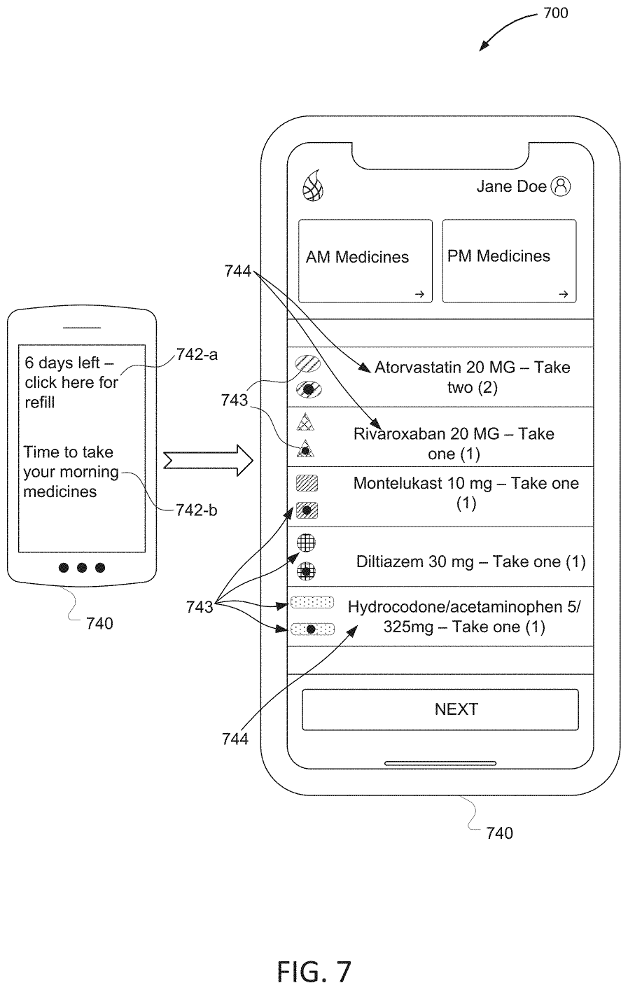

FIG. 7 illustrates an example of a process flow 700, according to various aspects of the disclosure. As shown in FIG. 7, a patient may receive one or more reminder notifications 742 on their mobile device 740. In some cases, when a reminder notification 742 (e.g., notification 742-a, notification 742-b) is selected, the mobile device 740 may display one or more images 743 of the actual pills along with an indication 744 of the name and/or number of pills (e.g., Atorvastatin 20 MG—take two, Rivaroxaban 20 MG—take one, etc.) that the patient is reminded to take. In some cases, the images 743 may comprise an image or photo of the front and back of the pills. For instance, in the example shown, the images 743 for each pill includes a visual depiction (e.g., a shape, such as ellipse for Atorvastatin, triangle for Rivaroxaban, rectangle with rounded edges for Singular, circle for Diltiazem, and an elongated rounded rectangle for Hydrocodone/acetaminophen) of the pill. Further, the images 743 with the black dot/circle represent images of the back of the pills. In some cases, the images 743 may also depict an imprint or label (if any) on the front and/or back sides of each pill, the color of the pill, etc. In some aspects, the visual depiction of the correct pill and dosage provides a simple and easily interpreted indication to help ensure proper dosing.

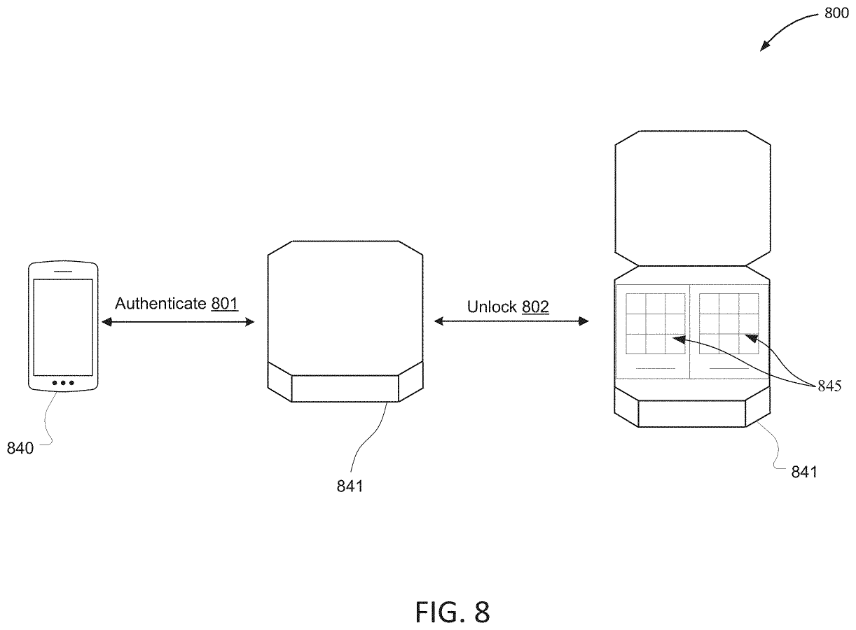

Turning now to FIG. 8, which illustrates a process flow 800 for authenticating and unlocking a smart medication security system 841, according to various aspects of the disclosure. In some cases, the smart medication security system 841 implements or more aspects of the smart medication security system 641, previously described in relation to FIG. 6. In some cases, process flow 800 may be implemented using a mobile device 840, where the mobile device 840 is in connection (e.g., using wireless means, such as Bluetooth, Wi-Fi, Near Field Communication or NFC) with the smart medication security system 841. The smart medication security system 841 may include a locking mechanism (e.g., a lid that is attached by hinges and secured by known electromechanical locking mechanisms to a base) that may be unlocked by the mobile device 840 (in response to an authentication process) to ensure that only the patient (or authorized user, e.g., a caregiver) has access to the pharmaceuticals.

In the example shown, the mobile device 840 may be used to authenticate 801 with the smart medication security system 841. In some cases, the authentication process may utilize two-factor authentication and/or biometric authentication such as finger, retinal, and/or facial recognition. For instance, a user or patient may need to open an application on their mobile device 840, where the application is associated with the smart medication security system 841, and input a PIN or passcode, provide a fingerprint scan or face ID, etc., to the application in order to unlock the security system 841. In some examples, the authentication process may be performed by the mobile device 840, the smart medication security system 841, or a combination thereof. For example, the mobile device 840 may transmit the information (e.g., PIN, passcode, etc.) input by the user to the smart medication security system 841, which then decides to authenticate or deny access to the user based on evaluating the received information. In other cases, the authentication 801 is performed at the mobile device, for instance, by the application stored on the mobile device 840. The mobile device 840 then transmits the authentication results (e.g., successful or unsuccessful) to the smart medication security system 841. If the authentication 801 is successful, the smart medication security system 841 unlocks 802 to allow the user/patient to retrieve the required medications.

In this way, the smart medication security system 841 helps to prevent narcotics and toxic medications from getting into children's hands while not being burdensome to patients. Medicines can be just as dangerous to children as a loaded gun, and they should be kept secure. Narcotics and addictive chemicals have the possibility of being diverted and stolen. These overdoses cause numerous deaths a year. The smart medication security system 841 helps to make sure medicine gets only to the people who need it, and only in the amounts needed. The smart medication security system 841 also helps ensure the wrong people don't get the dangerous medications.

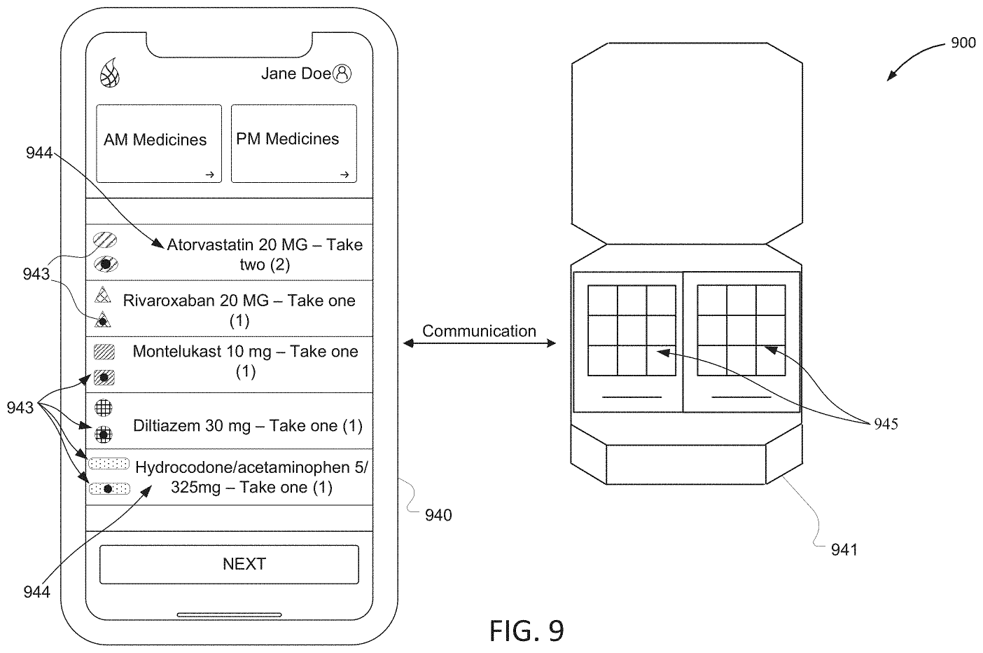

FIG. 9 illustrates a process flow 900, according to various aspects of the disclosure. As shown in FIG. 9, a locking smart medication security system 941 may include individual compartments 945 for each dose of a particular medication, and when the patient opens the security system 941, the compartment(s) 945 containing the medication(s) the patient should be taking (at that time) may be illuminated or otherwise identified to help prevent confusion about the medication that should be taken next. Additionally, or alternatively, a mobile device 940 in communication with the smart medication system 941 may display one or more images 943 of the actual pills along with an indication 944 of the name and/or number of pills (e.g., Atorvastatin 20 MG—take two, Rivaroxaban 20 MG—take one, etc.) that the patient is reminded to take. In some cases, the images 943 may comprise an image or photo of the front and back of the pills. For instance, in the example shown, the images 943 for each pill includes a visual depiction (e.g., a shape, such as ellipse for Atorvastatin, triangle for Rivaroxaban, rectangle with rounded edges for Montelukast, circle for Diltiazem, and an elongated rounded rectangle for Hydrocodone/acetaminophen) of the pill. Further, the images 943 with the black dot/circle represent images of the back of the pills. In some cases, the images 943 may also depict an imprint or label (if any) on the front and/or back sides of each pill, the color of the pill, etc. In some aspects, the visual depiction of the correct pill and dosage provides a simple and easily interpreted indication to help ensure proper dosing. In one non-limiting example, a user/patient may click or tap on the pill/medicine on the user device 940 and the smart medication system 941 may illuminate the compartment 945 containing the medicine, which may further enhance user experience. In some cases, once the user/patient has retrieved the medicine from the correct compartment, the application on the mobile device 940 may automatically update the list of medicines displayed on the device 940. For instance, the application may remove the medicine from the list or display a strikethrough over the text to prevent the user from accidentally taking more than the required dosage.

The smart medication security system 941 may be communicatively coupled to both a patient's mobile device (e.g., mobile device 940) and identified remote people/entities/devices via Wi-Fi connection and/or Bluetooth connection.

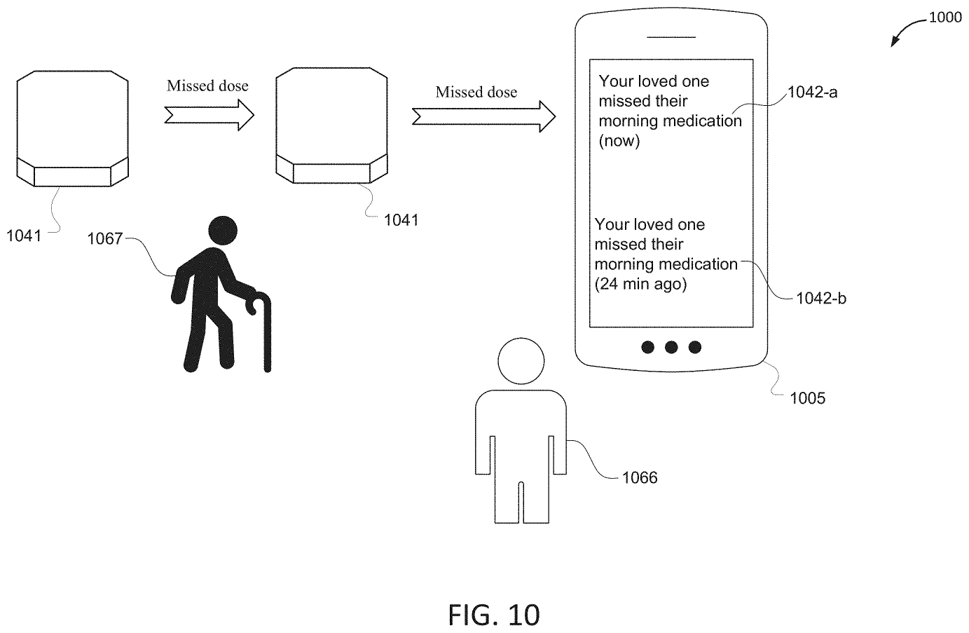

FIG. 10 illustrates a process flow 1000 to notify an individual 1066 (e.g., doctor, caregiver) when a patient 1067 has missed a dose, according to various aspects of the disclosure. Beneficially, as shown in FIG. 10, the network connection enables family members and/or doctors to be notified if a dose is missed. For example, the smart medication security system 1041 may initiate one or more notifications 1042 (e.g., notification 1042-a, notification 1042-b) if the locking lid is not opened by the patient 1067 for a threshold period of time. In some cases, the smart medication security system 1041 may automatically trigger an alert (i.e., notification 1042) that may be transmitted and displayed on the user device 1040 associated with the user 1066.

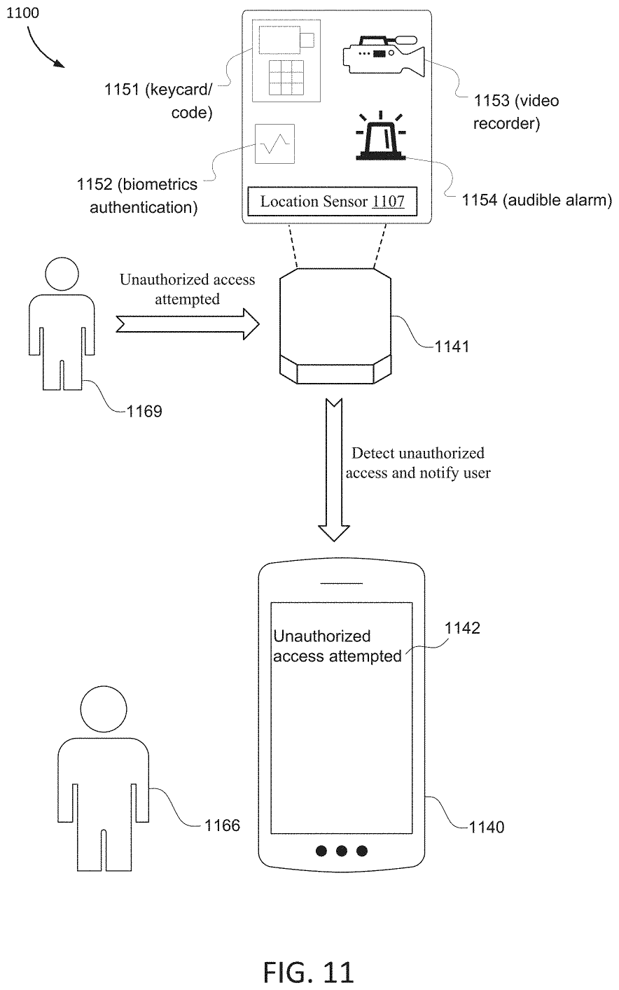

FIG. 11 illustrates a process flow 1100 for identifying and notifying a user 1166 when unauthorized access is attempted, according to an embodiment of the disclosure. In some cases, a notification and alarm may be triggered when unauthorized access is attempted. For example, incorrect entry of credentials (e.g., by a rogue or unauthorized user 1169) may trigger the alarm. It is also contemplated that the smart medication security system 1141 may have location sensing capabilities (Wi-Fi and/or GPS) that trigger an alarm (e.g., audible alarm 1154) if the smart medication security system 1141 is relocated. For instance, the smart medication security system 1141 may comprise one or more components, such as a keypad 1151 for entering a PIN or passcode, an imaging device 1153 capable of capturing images and/or video, a biometrics authentication system 1153 (e.g., for fingerprint recognition, voice recognition, retina or iris scan, facial recognition, etc.), an audible alarm 1154, and/or a location sensor 1107. In some embodiments, the smart medication security system may also include shock-detection capabilities (e.g., accelerometers and associated logic) to trigger the audible alarm 1154 if attempts are made to break into the smart medication security system 1141. As shown in process flow 1100, the smart medication security system 1141 may trigger a notification or alert 1142 for display on the user device 1140 (i.e., associated with the user/patient 1166) when it detects unauthorized access.

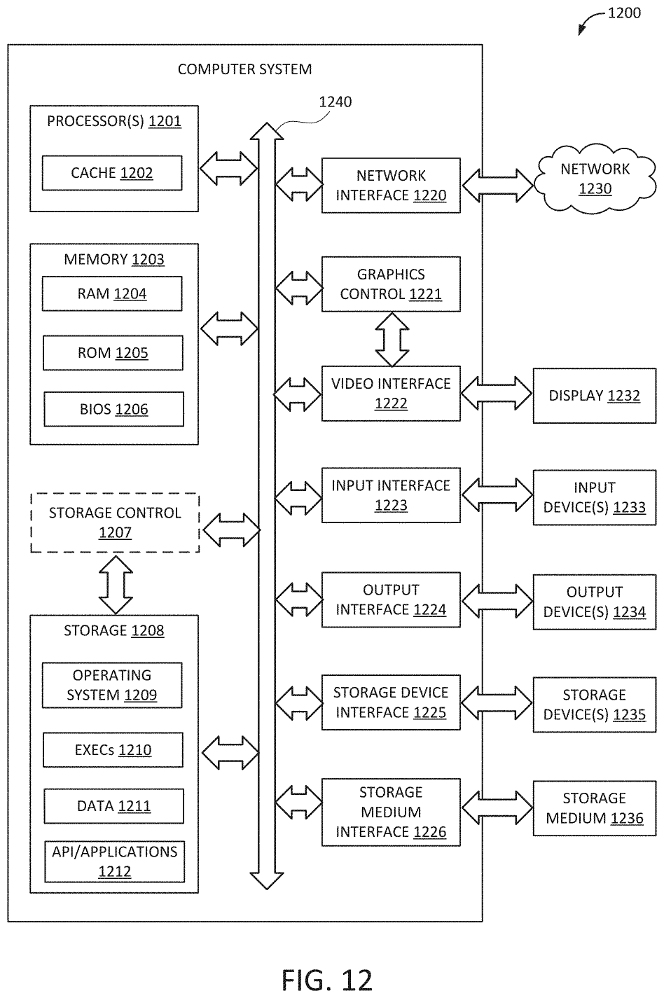

Referring to FIG. 12, it is a block diagram depicting an exemplary machine that includes a computer system 1200 within which a set of instructions can execute for causing a device to perform or execute any one or more of the aspects and/or methodologies of the present disclosure. For example, the exemplary machine may be utilized to realize aspects of the mobile devices described herein and aspects of the smart medication security system. The components in FIG. 12 are examples only and do not limit the scope of use or functionality of any hardware, software, embedded logic component, or a combination of two or more such components implementing particular embodiments.

Computer system 1200 may include a processor(s) 1201, a memory 1203, and a storage 1208 that communicate with each other, and with other components, via a bus 1240. The bus 1240 may also link a display 1232, one or more input devices 1233 (which may, for example, include a keypad, a keyboard, a mouse, a stylus, etc.), one or more output devices 1234, one or more storage devices 1235, and various tangible storage media 1236. All of these elements may interface directly or via one or more interfaces or adaptors to the bus 1240. For instance, the various tangible storage media 1236 can interface with the bus 1240 via storage medium interface 1226. Computer system 1200 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Processor(s) 1201 (or central processing unit(s) (CPU(s))) optionally contains a cache memory unit 1202 for temporary local storage of instructions, data, or computer addresses. Processor(s) 1201 are configured to assist in execution of computer readable instructions. Computer system 1200 may provide functionality for the components depicted in FIGS. 1-5 as a result of the processor(s) 1201 executing non-transitory, processor-executable instructions embodied in one or more tangible computer-readable storage media, such as memory 1203, storage 1208, storage devices 1235, and/or storage medium 1236. The computer-readable media may store software that implements particular embodiments, and processor(s) 1201 may execute the software. Memory 1203 may read the software from one or more other computer-readable media (such as mass storage device(s) 1235, 1236) or from one or more other sources through a suitable interface, such as network interface 1220. The software may cause processor(s) 1201 to carry out one or more processes or one or more steps of one or more processes described or illustrated herein. Carrying out such processes or steps may include defining data structures stored in memory 1203 and modifying the data structures as directed by the software.

The memory 1203 may include various components (e.g., machine readable media) including, but not limited to, a random-access memory component (e.g., RAM 1204) (e.g., a static RAM “SRAM”, a dynamic RAM “DRAM, etc.), a read-only component (e.g., ROM 1205), and any combinations thereof. ROM 1205 may act to communicate data and instructions unidirectionally to processor(s) 1201, and RAM 1204 may act to communicate data and instructions bidirectionally with processor(s) 1201. ROM 1205 and RAM 1204 may include any suitable tangible computer-readable media described below. In one example, a basic input/output system 1206 (BIOS), including basic routines that help to transfer information between elements within computer system 1200, such as during start-up, may be stored in the memory 1203.

Fixed storage 1208 is connected bidirectionally to processor(s) 1201, optionally through storage control unit 1207. Fixed storage 1208 provides additional data storage capacity and may also include any suitable tangible computer-readable media described herein. Storage 1208 may be used to store operating system 1209, EXECs 1210 (executables), data 1211, API applications 1212 (application programs), and the like. Often, although not always, storage 1208 is a secondary storage medium (such as a hard disk) that is slower than primary storage (e.g., memory 1203). Storage 1208 can also include an optical disk drive, a solid-state memory device (e.g., flash-based systems), or a combination of any of the above. Information in storage 1208 may, in appropriate cases, be incorporated as virtual memory in memory 1203.

In one example, storage device(s) 1235 may be removably interfaced with computer system 1200 (e.g., via an external port connector (not shown)) via a storage device interface 1225. Particularly, storage device(s) 1235 and an associated machine-readable medium may provide nonvolatile and/or volatile storage of machine-readable instructions, data structures, program modules, and/or other data for the computer system 1200. In one example, software may reside, completely or partially, within a machine-readable medium on storage device(s) 1235. In another example, software may reside, completely or partially, within processor(s) 1201.

Bus 1240 connects a wide variety of subsystems. Herein, reference to a bus may encompass one or more digital signal lines serving a common function, where appropriate. Bus 1240 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1200 may also include an input device 1233. In one example, a user of computer system 1200 may enter commands and/or other information into computer system 1200 via input device(s) 1233. Examples of an input device(s) 1233 include, but are not limited to, an alpha-numeric input device (e.g., a keyboard), a pointing device (e.g., a mouse or touchpad), a touchpad, a joystick, a gamepad, an audio input device (e.g., a microphone, a voice response system, etc.), an optical scanner, a video or still image capture device (e.g., a camera), and any combinations thereof. Input device(s) 1233 may be interfaced to bus 1240 via any of a variety of input interfaces 1223 (e.g., input interface 1223) including, but not limited to, serial, parallel, game port, USB, FIREWIRE, THUNDERBOLT, or any combination of the above.

In particular embodiments, when computer system 1200 is connected to network 1230, computer system 1200 may communicate with other devices, specifically mobile devices and enterprise systems, connected to network 1230. Communications to and from computer system 1200 may be sent through network interface 1220. For example, network interface 1220 may receive incoming communications (such as requests or responses from other devices) in the form of one or more packets (such as Internet Protocol (IP) packets) from network 1230, and computer system 1200 may store the incoming communications in memory 1203 for processing. Computer system 1200 may similarly store outgoing communications (such as requests or responses to other devices) in the form of one or more packets in memory 1203 and communicated to network 1230 from network interface 1220. Processor(s) 1201 may access these communication packets stored in memory 1203 for processing.

Examples of the network interface 1220 include, but are not limited to, a network interface card, a modem, and any combination thereof. Examples of a network 1230 or network segment 1230 include, but are not limited to, a wide area network (WAN) (e.g., the Internet, an enterprise network), a local area network (LAN) (e.g., a network associated with an office, a building, a campus or other relatively small geographic space), a telephone network, a direct connection between two computing devices, and any combinations thereof. A network, such as network 1230, may employ a wired and/or a wireless mode of communication. In general, any network topology may be used.

Information and data can be displayed through a display 1232. Examples of a display 1232 include, but are not limited to, a liquid crystal display (LCD), an organic liquid crystal display (OLED), a cathode ray tube (CRT), a plasma display, and any combinations thereof. The display 1232 can interface to the processor(s) 1201, memory 1203, and fixed storage 1208, as well as other devices, such as input device(s) 1233, via the bus 1240. The display 1232 is linked to the bus 1240 via a video interface 1222, and transport of data between the display 1232 and the bus 1240 can be controlled via the graphics control 1221.

In addition to a display 1232, computer system 1200 may include one or more other peripheral output devices 1234 including, but not limited to, an audio speaker, a printer, and any combinations thereof. Such peripheral output devices may be connected to the bus 1240 via an output interface 1224. Examples of an output interface 1224 include, but are not limited to, a serial port, a parallel connection, a USB port, a FIREWIRE port, a THUNDERBOLT port, and any combinations thereof.

In addition, or as an alternative, computer system 1200 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. An automatic desired volume of material visualization and display system, the system comprising:
an interface;
one or more hardware processors communicatively coupled to the interface, wherein the one or more hardware processors are configured by machine-readable instructions to:
receive a first measurement of a subject;
receive a desired material identifier;
receive a concentration of the desired material;
receive a use case scenario;
calculate a correct volume of the desired material, wherein such calculation is dependent upon:
the first measurement of the subject,
the desired material identifier,
the concentration of the desired material, and
the use case scenario;
retrieve, from a database, at least one image associated with the correct volume of the desired material; and
display the at least one image associated with the correct volume of the desired material on the interface.

2. The system of claim 1, wherein the one or more hardware processors are further configured by machine-readable instructions to:
receive one or more variables related to the subject; wherein,
the calculating is further based on the one or more variables; and
the desired material comprises a material associated with the subject and the use case scenario.

3. The system of claim 1, wherein the first measurement of the subject comprises a measurement indicative of at least of one of patient length and patient weight.

4. The system of claim 1, wherein the one or more hardware processors are further configured by machine-readable instructions to at least one of:
calculate one or more estimated values associated with the subject based on one or more formulas, and wherein the calculating of the correct volume is further based on the one or more estimated values;
receive one or more additional measurements;
receive the first measurement of the subject through a hardware measurement device.

5. The system of claim 1, wherein the one or more hardware processors are further configured by machine-readable instructions to display a written description of the correct volume adjacent to the at least one image.

6. The system of claim 1, wherein the one or more hardware processors are further configured by machine-readable instructions to send one or more pieces of information to an electronic medical records system.

7. A method for automatically displaying a visualization of a desired volume of material, the method comprising:
receiving a first measurement of a subject;
receiving an identifier of the desired material;
receiving a concentration of the desired material;
receiving a use case scenario;
calculating a correct volume of the desired material, wherein such calculation is dependent upon the first measurement of the subject, the desired material identifier, the concentration of the desired material, and the use case scenario;
retrieving, from a database, at least one image associated with the correct volume of the desired material; and
displaying the at least one image associated with the correct volume of the desired material.

8. The method of claim 7, further comprising:
receiving one or more variables related to the subject; and wherein
the calculating is further dependent upon the one or more variables.

9. The method of claim 7, further comprising:
calculating one or more estimated values associated with the subject based on one or more formulas, and wherein the calculating of the correct volume is further based on the one or more estimated values.

10. A computing platform configured for automatically displaying a visualization of a desired volume of material, the computing platform comprising:
a non-transient computer-readable storage medium having executable instructions embodied thereon; and
one or more hardware processors configured to execute the instructions to:
receive a first measurement of a subject for which a desired material is to be applied;
receive a name of the desired material receive a desired concentration of the desired material;
receive a use case scenario for an application of the desired material to the subject;
calculate, based on the first measurement of the subject, the name of the desired material, the desired concentration, and the use case scenario, a correct volume of the desired material for the application;
retrieve, from a database, at least one image associated with the correct volume of the desired material for the application; and
display, on an interface, the at least one image associated with the correct volume of the desired material.

11. A system configured for automatically displaying a visualization of a desired volume of material, the system comprising:
means for receiving a first measurement of a subject for which a desired material is to be applied;
means for receiving a name of the desired material receive a desired concentration of the desired material;
means for receiving a use case scenario for an application of the desired material to the subject;
means for calculating, based on the first measurement of the subject, the name of the desired material, the desired concentration, and the use case scenario, a correct volume of the desired material for the application;
means for retrieving, from a database, at least one image associated with the correct volume of the desired material for the application; and
means for displaying, on an interface, the at least one image associated with the correct volume of the desired material.

12. A non-transient computer-readable storage medium having instructions embodied thereon, the instructions being executable by one or more processors to perform a method for automatically displaying a desired volume of material, the method comprising:
receiving a first measurement of a subject;
receiving a name of the desired material receive a desired concentration of the desired material;
receiving a use case scenario for an application of the desired material to the subject;
calculating, based on the first measurement of the subject, the name of the desired material, the desired concentration, and the use case scenario, a correct volume of the desired material for the application;
retrieving, from a database, at least one image associated with the correct volume of the desired material for the application; and
displaying, on an interface, the at least one image associated with the correct volume of the desired material.

13. The computer-readable storage medium of claim 12, wherein the method further comprises:
receiving one or more variables related to the subject, and wherein the calculating is further based on the one or more variables.

14. The computer-readable storage medium of claim 12, wherein, the subject comprises a patient.

15. The computer-readable storage medium of claim 12, wherein the method further comprises receiving one or more additional measurements.

16. The computer-readable storage medium of claim 12, wherein the method further comprises receiving the first measurement of the subject through a hardware measurement device.

17. The computer-readable storage medium of claim 16, wherein the hardware measurement device is a smartphone camera.

18. The computer-readable storage medium of claim 14, wherein the desired material is a medication.

19. The computer-readable storage medium of claim 18, wherein,
the use case scenario comprises a medical use case scenario, and
the at least one image comprises of one of a syringe, and at least a portion of a tablet and a pill.

20. The computer-readable storage medium of claim 12, wherein the at least one image comprises an actual size of the correct volume.

21. The computer-readable storage medium of claim 20, wherein the at least one image comprises at least one of a color and a pattern, wherein the at least one of a color and a pattern is associated with at least one of,
the correct volume,
the desired material, and
a subject characteristic.

22. The system of claim 1 wherein the at least one image associated with the correct volume of the desired material on the interface comprises a pictogram.
